(19) United States
(12) Patent Application Publication
(10) Pub. No.: US 2025/0195126 A1
(43) Pub. Date: Jun. 19, 2025

(54) NEAR-CRITICAL ARGON BASED LOOP CATHETER FOR CIRCUMFERENTIAL ABLATION OF NERVE FIBERS

(71) Applicant: Medtronic Cryocath LP, Toronto (CA)

(72) Inventors: Jean-Pierre Lalonde, Pointe-Claire (CA); Nicolas Coulombe, Anjou (CA)

(21) Appl. No.: 18/847,848

(22) PCT Filed: Apr. 17, 2023
(86) PCT No.: PCT/CA2023/050514

§ 371 Date: Sep. 17, 2024

Related U.S. Application Data
(60) Provisional application No. 63/332,841, filed on Apr. 20, 2022.

(51) Int. Cl. A61B 18/02; A61B 18/00

(52) U.S. Cl. CPC A61B 18/02; A61B 2018/00017; A61B 2018/00023; A61B 2018/00404; A61B 2018/00434; A61B 2018/00541; A61B 2018/00577; A61B 2018/00642; A61B 2018/00839; A61B 2018/0212; A61B 2018/0268; A61B 2018/0287

(57) ABSTRACT

A method and near-critical argon-based loop catheter for circumferential ablation of nerve fibers are disclosed. According to one aspect, cryogenic catheter is configured to deliver near-critical-temperature argon cooling fluid to ablate parasympathetic innervation in nerve fibers in a wall of a passageway within a patient into which a distal portion of the cryogenic catheter is inserted, the passageway being one of an air passageway of a lung and a blood passageway. The cryogenic catheter includes at least one shaft to deliver argon cooling fluid to at least one expandable treatment element at the distal portion of the cryogenic catheter, an expandable treatment element being biased to form a multiple loop coil structure when expanded to make circumferential contact with the wall of the passageway. The cryogenic catheter also includes at least one expandable treatment element configured to be expandable by at least one of fluid pressure and mechanical force applied to a push wire within the shaft.

### CROSS-REFERENCE TO RELATED APPLICATION

The present application is related to and claims benefit under 35 U.S.C. § 119(e) from U.S. Provisional Patent Application Ser. No. 63/332,841, filed 20 Apr. 2022, entitled “NEAR-CRITICAL ARGON BASED LOOP CATHETER FOR CIRCUMFERENTIAL ABLATION OF NERVE FIBERS”, the entire contents of which being incorporated herein by reference.

### FIELD

The present technology is generally related to near-critical argon-based loop catheters for circumferential ablation of nerve fibers.

### BACKGROUND

Cryoablation is a technique that may be used with a catheter or other type of medical device to ablate tissue and has applications in, for example, cancer, nerve, and cardiac treatment. For example, cryoablation may be used to ablate parasympathetic innervation in the bronchi of a patient for treatment of chronic obstructive pulmonary disease (COPD), in the renal arteries for treatment of hypertension, and in the hepatic artery for treatment of hypertension and type 2 diabetes.

In some cryoablation systems, the catheter may be used to create lesions where heat is rapidly removed from cardiac cells, by delivering pressurized refrigerant, such as nitrous oxide (“N2O”), with a controlled mass flow rate, to the catheter. Heat may be transferred as the pressurized refrigerant expands and evaporates in the catheter tip. Cardiac cell lesions may be created via the rapid removal of heat. However, the temperatures attained by N2O may not be cold enough to cause permanent nerve block (i.e., the blocking of conduction of electrical signals by the treated nerve fibers.)

### SUMMARY

The techniques of this disclosure generally relate to near-critical argon-based loop catheters for circumferential ablation of nerve fibers.

According to one aspect, a cryogenic catheter is configured to deliver near-critical-temperature argon cooling fluid to ablate parasympathetic innervation in nerve fibers in a wall of a passageway within a patient into which a distal portion of the cryogenic catheter is inserted, the passageway being one of an air passageway of a lung and a blood passageway. The cryogenic catheter includes at least one shaft configured to deliver argon cooling fluid to at least one expandable treatment element at the distal portion of the cryogenic catheter. The cryogenic catheter also includes at least one expandable treatment element configured to receive the argon cooling fluid, the at least one expandable treatment element being biased to form a multiple loop coil structure when expanded to make circumferential contact with the wall of the passageway, an expandable treatment element being expandable by at least one of fluid pressure and mechanical force applied to a push wire within the shaft.

According to this aspect, in some embodiments, the at least one expandable treatment element is configured to form a plurality of approximately parallel multiple loop coil structures when expanded. In some embodiments, the at least one expandable treatment element is configured to be flexibly linear when not expanded. In some embodiments, the shaft is configured to deliver the argon cooling fluid through ports along a distal segment of the shaft. In some embodiments, at least one expandable treatment element includes a plurality of electrodes configurable to sense an electrical activity of nerve fibers in the wall of the passageway. In some embodiments, a temperature of the argon cooling fluid is below −100 degrees Celsius. In some embodiments, the push wire is configured to cause an expandable treatment element to retractably expand against the wall of the passageway, when the mechanical force is applied. In some embodiments, the shaft is configured to deliver the argon cooling fluid to an expandable treatment element via a Joule-Thomson valve. In some embodiments, the cryogenic catheter also includes a fluid supply lumen disposed within the shaft and extending within an expandable treatment element, the fluid supply lumen configured to deliver fluid to ports along a wall of the fluid supply lumen. In some embodiments, the cryogenic catheter also includes a reinforced guide sheath configured to encompass a portion of each of a plurality of shafts in a bundle.

According to another aspect, a cryogenic catheter is configured to deliver near-critical-temperature argon cooling fluid to ablate parasympathetic innervation in nerve fibers in a wall of a passageway within a patient into which a distal portion of the cryogenic catheter is inserted, the passageway being one of an air passageway of a lung and a blood passageway. The cryogenic catheter includes at least one shaft configured to deliver argon cooling fluid to at least one expandable treatment element at the distal portion of the cryogenic catheter, the at least one expandable treatment element being biased to form a multiple loop coil structure when expanded to make circumferential contact with the wall of the passageway. The cryogenic catheter also includes at least one expandable treatment element configured to be expandable by at least one of fluid pressure and mechanical force applied to a push wire within the shaft.

According to this aspect, in some embodiments, the multiple loop coil structure includes a helical coil having an axis approximately parallel to the wall of the passageway. In some embodiments, an expandable treatment element is configured to be flexibly linear when not inflated. In some embodiments, the shaft is configured to deliver the argon cooling fluid through ports along a distal segment of the shaft. In some embodiments, an expandable treatment element includes a plurality of electrodes configurable to sense an electrical activity of nerve fibers in the wall of the passageway. In some embodiments, a temperature of the argon cooling fluid below −100 degrees Celsius. In some embodiments, the push wire is configured to cause an expandable treatment element to retractably expand against the wall of the passageway, when the mechanical force is applied. In some embodiments, the shaft is configured to deliver the argon cooling fluid to an expandable treatment element via a Joule-Thomson valve. In some embodiments, the cryogenic catheter also includes a fluid supply lumen disposed within the shaft and extending within an expandable treatment element, the fluid supply lumen configured to deliver fluid to ports along a wall of the fluid supply lumen. In some embodiments, the cryogenic catheter also includes a reinforced guide sheath configured to encompass a portion of the shaft.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 shows a first configuration of a catheter and system constructed in accordance with the principles disclosed herein;

FIG. 2A shows a portion of an expandable treatment element of the system of FIG. 1 in a deflated state;

FIG. 2B shows the portion of the expandable treatment element of FIG. 2 in an inflated state;

FIG. 3 shows a distal portion of a second configuration of a catheter constructed in accordance with principles disclosed herein;

FIG. 4 shows an example embodiment of a cryogenic catheter with a push wire configured to expand a multiple loop coil structure; and

FIG. 5 shows circumferential expansion and lateral contraction of the coil shown in FIG. 4.

### DETAILED DESCRIPTION

Some embodiments include near-critical-temperature argon-based loop catheters for circumferential ablation of nerve fibers in a wall of a passageway within a patient, such as an air passageway of a lung or an artery. Some embodiments include using a multiple loop coil structure to deliver argon cooling fluid a more effective treatment to the tissue of the wall of the passageway. Use of argon cooling fluid enables a colder near-critical temperature of operation useful for ablation of parasympathetic innervation of nerve fibers in the wall of the air passage or artery. In some embodiments, the multiple loop coil structure is configured to make circumferential contact with the wall of the passageway, enabling an increased depth and length of the treatment area. In some embodiments, a push wire is configured to enable retractable expansion of the multi-loop coil structure to make contact with the wall of the passageway along a length of the wall. These and other features are disclosed in detail below.

Before describing in detail exemplary embodiments, it is noted that the embodiments reside primarily in combinations of apparatus components and processing steps related to near-critical argon-based loop catheters for circumferential ablation of nerve fibers in a wall of a passageway within a patient, such as an air passageway of a lung or an artery. Accordingly, components have been represented where appropriate by conventional symbols in the drawings, showing only those specific details that are pertinent to understanding the embodiments so as not to obscure the disclosure with details that will be readily apparent to those of ordinary skill in the art having the benefit of the description herein. Like numbers refer to like elements throughout the description.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a cryogenic catheter.

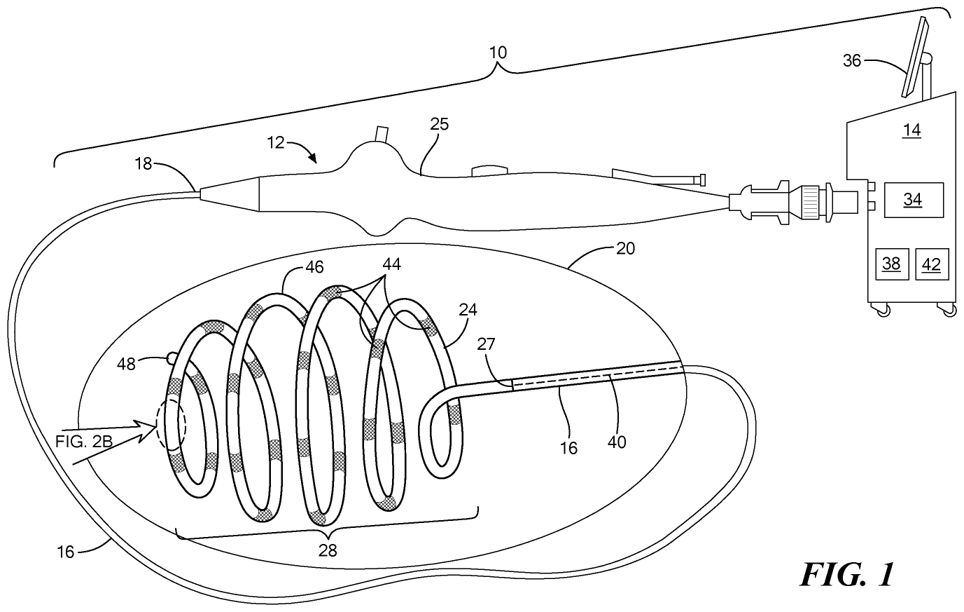

Referring now to the drawing figures where like elements have like reference numerals, FIG. 1 illustrates a medical system for circumferential ablation of nerve fibers and designated generally as “10”. The medical system 10 may include a cryogenic catheter 12 in electrical and/or fluid communication with a console 14. As shown in FIG. 1, the cryogenic catheter 12 may have a shaft 16 that includes proximal portion 18 and a distal portion 20 opposite the proximal portion 18. Although not shown in FIG. 1, the cryogenic catheter 12 may be used together with a second cryogenic catheter such as a guide sheath to assist in positioning the cryogenic catheter 12 within the lungs of the patient to circumferentially ablate nerve fibers. The shaft 16 of the cryogenic catheter 12 is sized and configured to be passable through a patient's vasculature or airways to be positioned proximate to an area of target nerve fibers within the lungs or within an artery, for example, to be ablated so that the ablated nerve fibers are blocked from conducting electrical signals.

The shaft 16 provides mechanical, electrical, and/or fluid communication between an expandable treatment element 24 and a handle 13 of the cryogenic catheter 12. The shaft 16 may be flexible to facilitate the navigation of the distal portion 20 and shaft 16 within the patient's body.

Additionally, the cryogenic catheter 12 further includes an expandable treatment element 24. As shown in FIG. 1, the expandable treatment element 24 is coupled to, and/or contiguous with, a distal portion 27 of the shaft 16 so that the expandable treatment element 24 may be passed through the patient's vasculature or airways towards an area of target tissue within the lungs, for example. The expandable treatment element 24 may also be flexible to allow for more desirable positioning proximate to an area of target nerve fibers. The expandable treatment element 24 may be a tube or sleeve made of memory shape material that is pre-shaped to match the contour of an inner surface of passageways of the lungs, such as the bronchi, or renal arteries and/or hepatic artery for treatment of hypertension or Type 2 diabetes. In some configurations, the expandable treatment element 24 may be a nitinol or polyimide injection tube covered with a thin polymer balloon sleeve. Nitinol is a metal alloy of nickel and titanium and exhibits elasticity and shape memory. More particularly, nitinol has an ability to undergo deformation in response to an applied force at a first temperature, retain the deformed shape at the first temperature when the applied force is removed, and then return to its former shape when heated to a second temperature.

In FIG. 1, the cryogenic catheter 12 is in electrical and/or fluid communication with the console 14. The console 14 includes one or more controllers, processors, and/or software modules containing instructions or algorithms to provide for the automated operation and performance of the features, sequences, or procedures described herein. In one embodiment, for example, the console 14 includes processing circuitry 34 programmed or programmable to execute the automated or semi-automated operation and performance of the features, sequences, calculations, or procedures described herein.

The processing circuitry 34 may include a memory and a processor. In particular, in addition to or instead of a processor, such as a central processing unit, and memory, the processing circuitry 34 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 34 may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory. The memory is in electrical communication with the processor and includes instructions that, when executed by the processor, configure the processor to receive, process, or otherwise use signals from the cryogenic catheter 12 and/or other system components. Still further, the console 14 may include one or more user input devices, controllers, speakers, and/or displays 36 for collection and conveying information from and to the user.

As shown in FIG. 1, the console 14 further includes a fluid supply reservoir 38 containing argon cooling fluid. The cryogenic catheter 12 includes a flexible fluid supply lumen 40 extending through a lumen defined by the shaft 16 and within the expandable treatment element 24. The flexible fluid supply lumen 40 is in fluid communication with the fluid supply reservoir 38 and/or console 14. The processing circuitry 34 is configured and/or programmed to initiate a delivery of argon cooling fluid from the fluid supply reservoir 38 to the cryogenic catheter 12 so that the expandable treatment element 24 may be expanded and cooled to remove heat from the tissue to be ablated. A fluid recovery reservoir 42 and/or scavenging system referenced to herein may be physically located within or external to the console 14. In one configuration, the fluid recovery reservoir 42 is configured for recovering or venting expended argon cooling fluid for re-use or disposal, and various control mechanisms. In addition to providing an exhaust function for the fluid supply, the console 14 may also include pumps, valves, controllers or the like to recover and/or re-circulate argon cooling fluid delivered to the shaft 16 and/or the fluid pathways of the system 10. Further, the console 14 may include a vacuum pump for creating a low-pressure environment in one or more conduits within the cryogenic catheter 12 so that refrigerant is drawn into the conduit(s)/lumen(s) of the shaft 16. However, as mentioned above, the fluid supply reservoir 38, the fluid recovery reservoir 42, or scavenging system may instead be separate from, but in communication with, the console 14.

A plurality of electrodes 44 may be disposed along, coupled to, or otherwise printed on the outer surface 46 of the expandable treatment element 24. As shown in FIG. 1, the expandable treatment element has a distal end 48. In some embodiments, each electrode of the plurality of electrodes 44 may be a ring electrode or button electrode printed or coupled to the outer surface 46 of the expandable treatment element 24. Each electrode 44 may be in electrical communication with a flexible tracing or wire (not shown) that is in electrical communication with the console 14. Alternatively, more than one electrode 44 may be in communication with a single tracing or wire. Also, in some embodiments, the plurality of electrodes 44 may be uniformly spaced apart along the outer surface 46 of the expandable treatment element 24. The plurality of electrodes 44 may also be positioned such that a distance between each electrode 44 in a first pair of adjacent electrodes is different than a distance between each electrode 44 in a second pair of adjacent electrodes 44. In other words, in some configurations, the spacing between electrodes 44 may be uniform, and in other configurations, the spacing between electrodes 44 may not be uniform. The plurality of electrodes 44 may be flexible, stretchable, and/or may not be cinched down on the outer surface of the expandable treatment element 24. In some such configuration, the electrodes 44 and/or the tracings may be printed in a zig-zag, spiral, helical, radial, or offset pattern along the length of the expandable treatment element 24.

In some embodiments, the expandable treatment element 24 may be biased to form a coil structure that when expanded, makes circumferential contact with a wall of a passageway. The expandable treatment element 24 may be caused to expand by at least one of fluid pressure and mechanical force applied to a push wire, for example, within the shaft 16. As noted above, the temperatures attained by N2O may not be cold enough to cause permanent nerve block (i.e., the blocking of conduction of electrical signals by the treated nerve fibers.) In contrast, Argon can achieve much lower cooling temperatures, below −100 degrees Celsius to achieve permanent nerve block in applications where N2O is not cold enough.

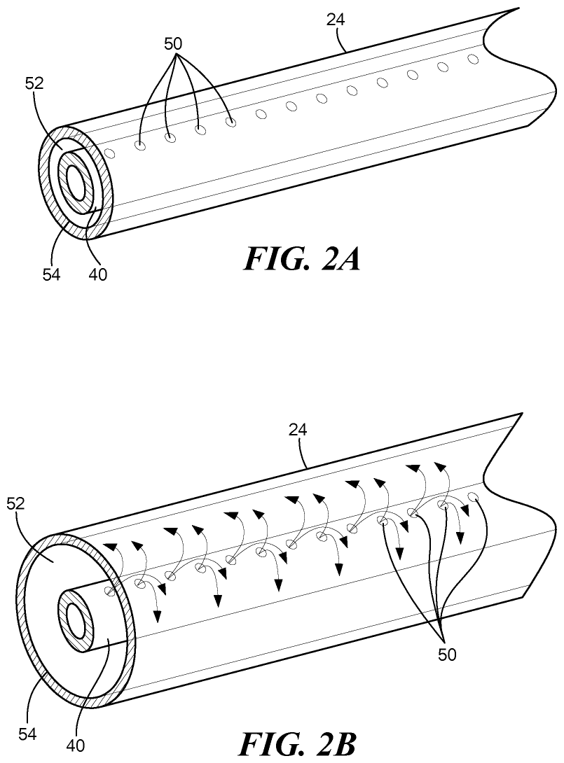

Now referring to FIGS. 2A-2B, the expandable treatment element 24 includes the flexible fluid supply lumen 40 disposed therein. The flexible fluid supply lumen 40 may define a plurality of injection orifices, or ports 50 to facilitate the dispersion of argon cooling fluid towards an interior 52 of the expandable treatment element 24, the interior being bound by inner surface 54. In some configurations, as shown in FIG. 2A, the expandable treatment element 24 is in an unexpanded configuration. When in the unexpanded configuration, the expandable treatment element 24 may be more easily navigated within and through the patient's body and into a bronchi of the lungs, for example. The unexpanded configuration allows the expandable treatment element 24 to be more freely maneuvered through the air passageway or artery and into a desired position for treatment. Accordingly, the flexible fluid supply lumen 40 disposed within the expandable treatment element 24 may be configured to bend to conform to the curvature of the expandable treatment element 24.

As argon cooling fluid is dispersed towards the inner surface 52 of the expandable treatment element 24, the dispersed argon cooling fluid aggregates within an inner chamber defined between the flexible fluid supply lumen 40 and the inner surface 52 of the expandable treatment element 24. As more argon cooling fluid is collected within the interior of the expandable treatment element 24, the expandable treatment element 24 may expand such that the diameter of the multiple loop coil structure 28 formed by the expandable treatment element 24 increases (as shown in FIG. 2B). In other words, the expandable treatment element 24 may be caused to transition between an unexpanded configuration and an expanded configuration, and vice versa. The expandable treatment element 24 may be caused to return to the unexpanded configuration by suctioning argon cooling fluid from the interior of the expandable treatment element 24. The suction may be caused by a vacuum source or pump (not shown) disposed within the console 14, or in communication with the console 14. The suctioned argon cooling fluid then travels between a gap, chamber, or space defined between the inner wall of the shaft 16 and the flexible fluid supply lumen 40, in some embodiments. The argon cooling fluid may be suctioned to a fluid recovery reservoir and/or scavenging system disposed within, or external to, the console 14.

Continuing to refer to FIG. 1 and FIGS. 2A-2B, the expandable treatment element 24 can be biased to a spiral, helical, or otherwise coiled shape, which can be predefined, or the expandable treatment element 24 may be caused to expand by the application of force and/or by inflating the expandable treatment element 24. When in the expanded configuration, the expandable treatment element 24 may also define a multiple loop coil structure 28 having a plurality of loops (shown in FIG. 1) that are sized and configured to be in contact with a wall of the patient's bronchi or other air passageway, or to make contact with the wall of an artery. Following the completion of an ablation procedure or other cryogenic procedure, the dispersed argon cooling fluid may pass from the inner chamber of the expandable treatment element 24, through the lumen of the shaft 16 towards the proximal portion 18, and to a fluid recovery reservoir 42 and/or scavenging system so that the expandable treatment element 24 can return to its deflated free-form configuration.

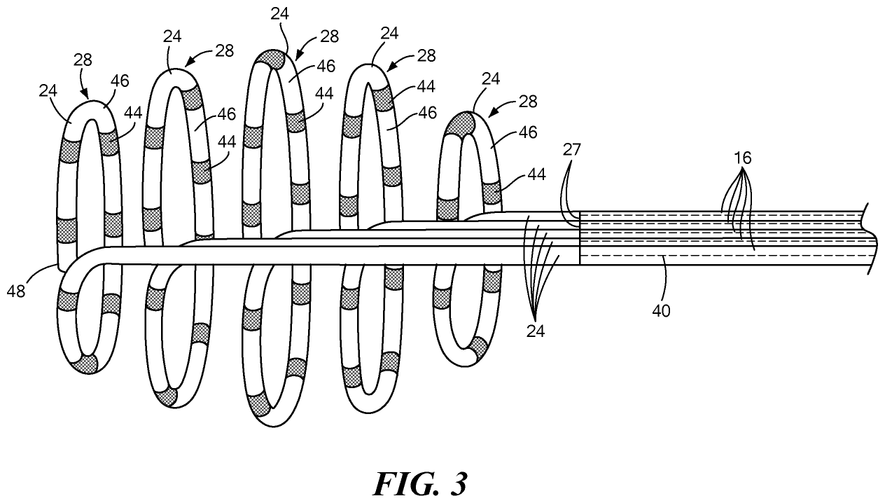

Now referring to FIG. 3, the cryogenic catheter 12 includes a plurality of shafts 16 and a plurality of expandable treatment elements 24 coupled to and/or contiguous with the distal portion 27 of each respective shaft 16 to have an enhanced cooling distribution of refrigerant. In some configurations, each shaft 16 of the plurality of shafts is coupled, adhered, or otherwise bonded to at least one adjacent shaft 16. In other configurations, not shown, the plurality of shafts 16 may be disposed within a lumen having a plurality of isolated channels each sized and configured to receive one shaft 16 of the plurality of shafts. Each individual expandable treatment element 24 can receive the argon cooling fluid.

In some embodiments, the cryogenic catheter 12 may include five expandable treatment elements 24 and five shafts 16. Each shaft 16 may have a flexible fluid supply lumen 40 that extends through a respective expandable treatment element 24 coupled to, and/or contiguous with, the distal portion 27 of the shaft 16 and is in communication with the console 14. Each expandable treatment element 24 can be caused to expand to an expanded configuration and contract to an unexpanded configuration. Although five expandable treatment elements 24 are shown in FIG. 3, more or less than five expandable treatment elements 24 may be employed, as deemed necessary by the clinician to achieve a desired ablative efficacy or pattern. As shown in FIG. 3, when in the expanded configuration, each expandable treatment element 24 may define at least one loop 28 or spiral that is sized and configured to approximate or substantially match the curvature of the air passageway or artery, so that the expanded treatment element 24 enlarges circumferentially to make contact with the wall of the air passageway or artery. In some embodiments, the features of making circumferential contact with the walls of the passageway by multiple loops of a multiple loop coil structure may enable the clinician to optimize the depth of tissue affected by the cryogenic treatment at very low temperatures by increasing the length of the cooling area. This may have the effect of shielding a larger portion of tissue from surrounding heat. The ability to shield larger portions of tissue from surrounding heat may enable attainment of very low temperatures, for example, less than −100 degrees Celsius, which is lower than an operating temperature of about −80 degrees Celsius of cooled N2O.

Continuing to refer to FIG. 3, each expandable treatment element 24 may be spaced apart from an adjacent expandable treatment element 24 so that no two expandable treatment elements are in physical contact with each other. Further, the expandable treatment elements 24 may be inflated such that each expandable treatment element 24 has a diameter that is the same as or different than an outer diameter of another expandable treatment element 24. For example, one respective loop may have an outer diameter that is larger or smaller than the outer diameter of the other remaining loops of the multiple loop coil structure 28 formed by the plurality of expandable treatment elements 24 shown in the example of FIG. 3. In some embodiments, expandable treatment elements 24 having different diameters may enable accommodation of the variable diameter of bronchi, for example.

As shown in FIGS. 1 and 3, each expandable treatment element 24 includes a plurality of electrodes 44 in communication with the console 14 and configured to monitor a quality, level, or degree of contact between each expandable treatment element 24 and the area of target tissue within the lung passageway and further detect the growth of any ice on the target tissue during a cryoablation procedure. Cryoablation may be referred to as the treatment of target tissue with thermal energy, and in particular, involves delivering argon cooling fluid to the expandable treatment element 24 at a low enough temperature to extract heat from the target tissue to ablate parasympathetic innervation in the walls of the air passageway or artery.

Before, during or after a cryoablation procedure, a plurality of the electrodes 44 may deliver test signals to the area of target tissue and subsequently receive response signals that indicate biological electrical activity within the area of target tissue in response to the test signals. The received response signals may be conducted from the electrodes 44 to the console 14. A memory associated with the processing circuitry 34 is configured to store received response signals once they are digitized by the processing circuitry 34 and may be used as received or used subsequently by the processing circuitry 34 to determine a degree of tissue contact between the expandable treatment element 24 and a wall of the air passageway or artery, based at least in part on the received response signal. This information may be relayed to the patient and/or clinician via the 36 display, computer monitor, smartphone screen, or the like.

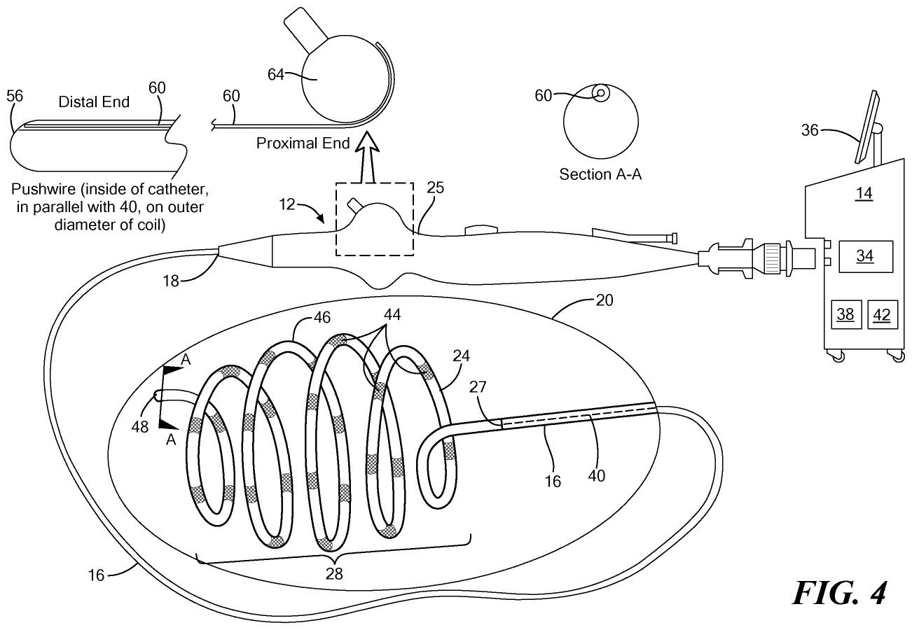

FIG. 4 illustrates an example embodiment where the expandable treatment element 24 is configured with a push wire 60 that is internal to the expandable treatment element 24 and that extends to the distal end 48 of the expandable treatment element 24. This enables the argon cooling fluid to reach the distal end 48. In some embodiments, the argon cooling fluid may be injected into a port at the distal end 48 of the expandable treatment element 24 to expand the expandable treatment element and to apply therapy to the nerve fibers in the wall of the passageway.

Within the handle 13 of the device within region 62 is a mechanical switch 64 to which the push wire 60 is connected. When the switch 64 is in a first position, the push wire 60 is in a retracted position so that it does not expand the expandable treatment element 24. In a second position of the switch 64, the push wire 60 is extends to an extended position to expand the expandable treatment element 24 to an expanded configuration. The push wire 60 may be retracted from the extended configuration by the switch 64, thereby returning the expandable treatment element to an unexpanded configuration. In some embodiments, the switch 64 may include a spring to communicate a force applied to a handle of the switch to the push wire 60 and to maintain the push wire 60 in an extended configuration to maintain the expandable treatment element 24 in the expanded configuration.

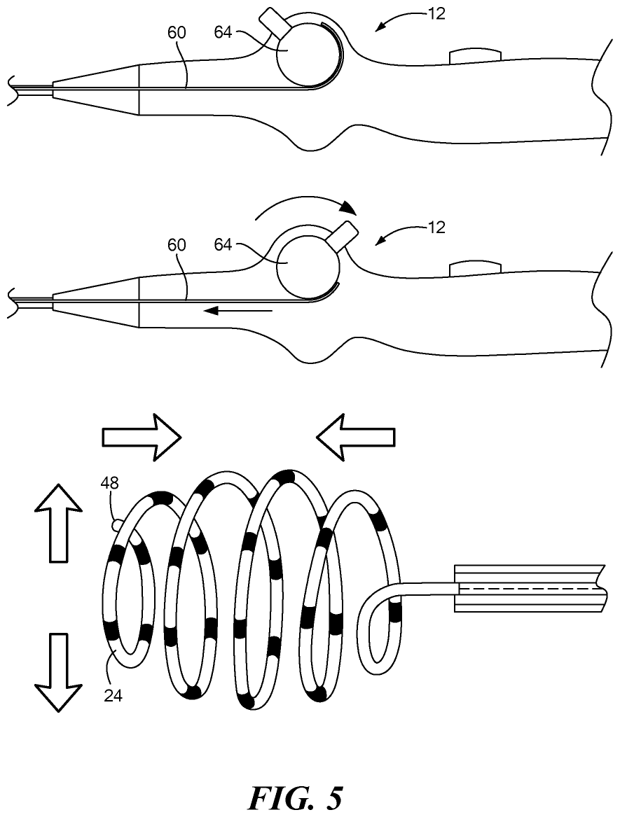

FIG. 5 is a diagram shown directions of circumferential expansion and lateral contraction when the switch 64 is moved into the second position, i.e., when the push wire 60 is in an extended position. In embodiments involving multiple separate loops such as shown in FIG. 3, there may be one push wire 60 for each loop, each of which may be controlled by a single switch 64 or by multiple switches 64. In some embodiments, an expandable treatment element 24 may be expanded by a combination of mechanical force applied to the push wire 60 and by fluid pressure of the argon cooling fluid applied to the expandable treatment element 24. In some embodiments, the combination of mechanical force and fluid pressure may enable a more controlled and full expansion of the expandable treatment element 24. Also, the application of mechanical force prior to applying the fluid may be desirable to assess quality of contact prior to application of thermal energy.

As shown in FIGS. 4 and 5, an embodiment of the cryogenic catheter 12 is configured to deliver near-critical-temperature argon cooling fluid to ablate parasympathetic innervation in nerve fibers in a wall of a passageway within a patient into which a distal portion 20 of the cryogenic catheter 12 is inserted, the passageway being one of an air passageway of a lung and a blood passageway. The cryogenic catheter 12 includes a shaft 16 configured to deliver argon cooling fluid to at least one expandable treatment element 24 at the distal portion 20 of the cryogenic catheter 12. The cryogenic catheter 12 also includes at least one expandable treatment element 24 configured to receive the argon cooling fluid, an expandable treatment element 24 being biased to form a multiple loop coil structure 28 when expanded to make circumferential contact with the wall of the passageway, an expandable treatment element 24 being expandable by at least one of fluid pressure and mechanical force applied to a push wire 60 within the shaft 16.

According to this aspect, in some embodiments, the at least one expandable treatment element 24 is configured to form a plurality of approximately parallel coil structures 28 when expanded. In some embodiments, the at least one expandable treatment element 24 is configured to be flexibly linear when not expanded. Flexibly linear means that the expandable treatment element 24 may extend linearly in a direction and also be flexed to assume a non-linear disposition.

In some embodiments, the shaft 16 is configured to deliver the argon cooling fluid through ports along a distal segment of the shaft 16. In some embodiments, at least one expandable treatment element 24 includes a plurality of electrodes configurable to sense an electrical activity of nerve fibers in the wall of the passageway. In some embodiments, a temperature of the argon cooling fluid below −100 degrees Celsius. In some embodiments, the push wire 60 is configure to cause an expandable treatment element 24 to retractably expand against the wall of the passageway, when the mechanical force is applied. Retractably expanding means that the expandable treatment element may be expanded to expand outward or inward in a radial direction (normal to a center axis of the expandable treatment element 24.

In some embodiments, the shaft 16 is configured to deliver the argon cooling fluid to an expandable treatment element 24 via a Joule-Thomson valve. In some embodiments, the cryogenic catheter 12 also includes a fluid supply lumen disposed within the shaft 16 and extending within an expandable treatment element 24, the fluid supply lumen configured to deliver fluid to ports along a wall of the fluid supply lumen. In some embodiments, the cryogenic catheter 12 also includes a reinforced guide sheath configured to encompass a portion of each of a plurality of shafts 16 in a bundle.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a cryogenic catheter 12.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope of the following claims.

What is claimed is:

1. A cryogenic catheter configured to deliver near-critical-temperature argon cooling fluid to ablate parasympathetic innervation in nerve fibers in a wall of a passageway within a patient into which a distal portion of the cryogenic catheter is inserted, the passageway being one of an air passageway of a lung and a blood passageway, the cryogenic catheter comprising:
at least one shaft configured to deliver argon cooling fluid to at least one expandable treatment element at the distal portion of the cryogenic catheter;
at least one expandable treatment element configured to receive the argon cooling fluid, the at least one expandable treatment element being biased to form a coil structure when expanded to make circumferential contact with the wall of the passageway, an expandable treatment element being expandable by at least one of fluid pressure and mechanical force applied to a push wire within the shaft.

2. The cryogenic catheter of claim 1, wherein the at least one expandable treatment element is configured to form a plurality of approximately parallel coil structures when expanded.

3. The cryogenic catheter of claim 1, wherein the at least one expandable treatment element is configured to be flexibly linear when not expanded.

4. The cryogenic catheter of claim 1, wherein the shaft is configured to deliver the argon cooling fluid through ports along a distal segment of the shaft.

5. The cryogenic catheter of claim 1, wherein at least one expandable treatment element includes a plurality of electrodes configurable to sense an electrical activity of nerve fibers in the wall of the passageway.

6. The cryogenic catheter of claim 1, wherein a temperature of the argon cooling fluid below −100 degrees Celsius.

7. The cryogenic catheter of claim 1, wherein the push wire is configured to cause an expandable treatment element to retractably expand against the wall of the passageway, when the mechanical force is applied.

8. The cryogenic catheter of claim 1, wherein the shaft is configured to deliver the argon cooling fluid to an expandable treatment element via a Joule-Thomson valve.

9. The cryogenic catheter of claim 1, further comprising a fluid supply lumen disposed within the shaft and extending within an expandable treatment element, the fluid supply lumen configured to deliver fluid to ports along a wall of the fluid supply lumen.

10. The cryogenic catheter of claim 1, further comprising a reinforced guide sheath configured to encompass a portion of each of a plurality of shafts in a bundle.

11. A cryogenic catheter configured to deliver near-critical-temperature argon cooling fluid to ablate parasympathetic innervation in nerve fibers in a wall of a passageway within a patient into which a distal portion of the cryogenic catheter is inserted, the passageway being one of an air passageway of a lung and a blood passageway, the cryogenic catheter comprising:
at least one shaft configured to deliver argon cooling fluid to at least one expandable treatment element at the distal portion of the cryogenic catheter, the at least one expandable treatment element being biased to form a multiple loop coil structure when expanded to make circumferential contact with the wall of the passageway;
at least one expandable treatment element configured to be expandable by at least one of fluid pressure and mechanical force applied to a push wire within the shaft.

12. The cryogenic catheter of claim 11, wherein the multiple loop coil structure includes a helical coil having an axis approximately parallel to the wall of the passageway.

13. The cryogenic catheter of claim 11, wherein an expandable treatment element is configured to be flexibly linear when not inflated.

14. The cryogenic catheter of claim 11, wherein the shaft is configured to deliver the argon cooling fluid through ports along a distal segment of the shaft.

15. The cryogenic catheter of claim 11, wherein an expandable treatment element includes a plurality of electrodes configurable to sense an electrical activity of nerve fibers in the wall of the passageway.

16. The cryogenic catheter of claim 11, wherein a temperature of the argon cooling fluid below −100 degrees Celsius.

17. The cryogenic catheter of claim 11, wherein the push wire is configured to cause an expandable treatment element to retractably expand against the wall of the passageway, when the mechanical force is applied.

18. The cryogenic catheter of claim 11, wherein the shaft is configured to deliver the argon cooling fluid to an expandable treatment element via a Joule-Thomson valve.

19. The cryogenic catheter of claim 11, further comprising a fluid supply lumen disposed within the shaft and extending within an expandable treatment element, the fluid supply lumen configured to deliver fluid to ports along a wall of the fluid supply lumen.

20. The cryogenic catheter of claim 11, further comprising a reinforced guide sheath configured to encompass a portion of the shaft.
